(19) United States
(12) Patent Application Publication
(10) Pub. No.: US 2019/0388831 A1
(43) Pub. Date: Dec. 26, 2019

(54) DEVICE FOR SEPARATING CONDENSED WATER

(71) Applicant: Berlin Heart GmbH, Berlin (DE)

(72) Inventors: Nedim Arslan, Berlin (DE); Kurt Graichen, Berlin (DE); Christian Robbenmenke, Berlin (DE); Aaron Rodemerk, Potsdam (DE)

(73) Assignee: Berlin Heart GmbH, Berlin (DE)

(21) Appl. No.: 16/480,739

(22) PCT Filed: Jan. 24, 2018
(86) PCT No.: PCT/EP2018/051753

§ 371 Date: Jul. 25, 2019

(30) Foreign Application Priority Data
Jan. 26, 2017 (EP) 17153346.6

(51) Int. Cl. B01D 53/26; A61M 1/10; B01D 5/00

(52) U.S. Cl. CPC B01D 53/265; A61M 1/1096; A61M 2205/3331; A61M 1/106; B01D 5/009; A61M 1/1008

(57) ABSTRACT

A device for separating condensed water from an operating gas of a pneumatic pulsatile system is provided. The device has a gas line section with a gas inlet and a first and a second gas outlet. The device further comprises a condensation container for extracting the condensed water out of the operating gas, said condensation container being connected to the second gas outlet. A transition region from the second gas outlet to the condensation container has a cross-sectional enlargement, and the condensation container has means for discharging the extracted condensed water. A pneumatic pulsatile system and a cardiac support system are also provided.

### CROSS-REFERENCE TO RELATED APPLICATIONS

This application is a 371 nationalization of international patent application PCT/EP2018/051753 filed Jan. 24, 2018, which claims priority under 35 USC § 119 to European patent application EP 17153346.6 filed Jan. 26, 2017. The entire contents of each of the above-identified applications are hereby incorporated by reference.

### BRIEF DESCRIPTION OF THE DRAWINGS

The figures are as follows

FIG. 1 a schematic representation of an inventive device for separating condensed water according to a first sample embodiment;

FIG. 2 a schematic representation of an inventive device for separating condensed water according to a second sample embodiment;

FIG. 3 a test setup to prove the efficacy of the inventive device; and

FIG. 4 a diagram to illustrate the time behavior of the amount of condensed water separated in the test setup from FIG. 3.

### DETAILED DESCRIPTION

This invention relates to a device for separating condensed water from an operating gas of a pneumatic system, especially a pneumatic pulsatile system for a diaphragm pump of a cardiac support system.

Such cardiac support systems are provided for left side or right side or bilateral cardiac support when the natural heart is unable, even under drug therapy or due to invasive intensive care measures, to maintain a blood flow and blood pressure that are adequate for the patient. Each of these systems consists of one or two diaphragm pumps (also referred to below as blood pumps or heart pumps) with drive tubes and cannulas and a drive system (pneumatic pulsatile system), and are intended for use in the clinical and home environment. Furthermore, the systems are intended for mid- to long-term mechanical support.

The blood pump consists of a blood chamber and an air chamber, which are separated from one another by a flexible multilayer membrane made of polyurethane. A drive tube connects the air chamber of the blood pump with the drive. The suction pressure or driving pressure produced by the pneumatic drive moves the membrane, filling and emptying the blood pump. The blood flows out of the atrium or ventricle through the inlet cannula into the blood chamber of the blood pump, and from there through the vascular cannula into the pulmonary artery or the aorta. Valves in the inlet or outlet area of the blood pump ensure a directed blood flow. In the pneumatic drive, negative pressure and excess pressure alternately build up against the membrane of the blood pump, filling and emptying the blood pump. The pressure can be produced in different ways. For example, a motor can move a piston back and forth in a cylinder through a rod. This piston movement produces the alternate negative pressure and excess pressure. An equalizing valve controls the air mass in the cylinder. Another drive mechanism is designed so that two containers are constantly supplied, by corresponding compressors, with negative pressure and excess pressure, and a special valve is used which alternately connects, through a drive tube, the diaphragm pump with one of the two containers.

When air is compressed, for example in piston compressors for producing compressed air, water can come out of it in the form of condensate. Condensed water can also arise when cold surfaces come in contact with warm, humid air. Both effects also occur in the above-described pneumatic pulsatile systems for cardiac support systems. Previously known pneumatic systems have no satisfactory mechanism to discharge the condensed water that arises.

In these pneumatic pulsatile systems, the condensed water preferably collects, for example, in the pneumatic tubes between the drive piston and the diaphragm pump. If condensed water forms, the tubes must be disconnected and the water must be manually drained. From time to time, these circumstances can disturb and be a burden to the patient that is connected to the device.

In some systems, condensed water could be observed inside the drive in some cases. In a supply line from the main pneumatic leg to a pressure sensor, the condensate can functionally impair the sensor.

Conventional condensed water separators are available, which are principally used in the production of compressed air. These separators have the disadvantage that they require a great deal of space, that some models consume additional energy, and that a potential source of unwanted leakage (in the case of malfunction) is integrated into the system.

Also known are passive systems, which work with a tube that is airtight, but allows water vapor to diffuse through it. However, to allow diffusion, high pressure differences (about 4-5 bar) are necessary.

Therefore, one goal of this invention is to provide a device for separating condensed water which solves the above-described problems of the prior art. In particular, it is a goal of this invention to provide a device that allows effective condensed water separation, requires no additional energy, is very compact, and presents a very small risk of leakage. It is another goal to provide a pneumatic pulsatile system and a cardiac support system with such a device.

The inventive device for separating condensed water from an operating gas of a pneumatic system has a gas line section that is connected with the pneumatic system and that has a gas inlet and a first and a second gas outlet. This gas line section is connectable, at its gas inlet and its first gas outlet, with a gas line for carrying an operating gas, preferably air, of a pneumatic system, so that the operating gas can get into the gas line section through the gas inlet and can come out of the gas line section through the first gas outlet.

The inventive device further comprises a condensation container for extracting the condensed water from the operating gas and for collecting the condensed water. To discharge the condensed water, the condensation container has a water-permeable material, especially a semipermeable plastic, a fluoropolymer, and/or a sintered metal.

The water-permeable material of the condensation container, such as, in particular, a semipermeable plastic, fluoropolymer, or sintered metal, allows the condensed water to diffuse out of the condensation container, and thus discharge of the condensed water from the condensation container. It is especially preferable if the water-permeable material of the condensation container is impermeable to gas, to prevent leakage of the operating gas. Furthermore, it is especially advantageous if the material can be continually wetted with water. In this case, the condensed water can be discharged even at low pressures. It was possible to confirm the efficacy of this principle experimentally.

In order to extract and collect the condensed water more effectively, a transitional area from the second gas outlet to the condensation container has a cross-sectional enlargement. This is done by exploiting a thermodynamic effect that is described below. The pneumatic drive produces a cyclic excess pressure or negative pressure, which the gas line communicates to a diaphragm pump that is connected with the gas line. As the operating gas transitions from the gas line to the pump, it expands rapidly, and a cooling in comparison with the ambient temperature is observed. For this reason, the water held in the operating gas preferably condenses at this place. The same effect occurs, for example, when pressure sensors are connected with the pneumatic pulsatile system (also referred to as a pneumatic or drive line), causing changes in cross section at the transition from the gas line to the pressure sensor. The invention deliberately exploits this principle by shaping the transitional area from the gas outlet to the condensation container with a cross-sectional enlargement. In this way, the condensed water is collected in the condensation container and cannot condense in unwanted places. It was confirmed in an experiment that the principle is functional and that the condensed water arises principally in the condensation container provided for this purpose.

The inventive device can be used especially in stationary and non-stationary pneumatic drives for cardiac support systems, for reliable prevention of an accumulation of condensed water. The device works completely independently and is, for the most part, maintenance-free, so that it offers high added value for the patient. It is also conceivable to use the invention in other systems in which pulsatile pressure conditions prevail and condensed water should be discharged. For example, it is conceivable to use the system in tubes for patients receiving artificial respiration.

In one advantageous embodiment of the invention, the condensation container can have a wall, at least one subarea of which contains or consists of a water-permeable material, in particular a semipermeable plastic, a fluoropolymer, and/or a sintered metal. The size of this subarea in relation to the size of the surface of the wall is generally dependent on the material used and the amount of water to be discharged. In a preferred embodiment of the invention, the size of the subarea with the water-permeable material is about 5 cm².

In an alternative embodiment of the invention, a valve or a pump can be arranged in the condensation container to provide the means for discharging the condensed water. It would also be conceivable to connect the condensation container with the second gas outlet through a separable coupling.

To intensify the condensation effect, the transitional area from the second gas outlet to the condensation container can have a cross-sectional narrowing before the cross-sectional enlargement. This will compress the operating gas flowing through the transitional area before it expands. Compressing the operating gas allows at least part of the water vapor held in the operating gas to condense, and thus increases the amount of water extracted from the operating gas. In particular, it is possible to arrange, between the second gas outlet and the condensation container, a gas line connecting piece, whose minimum cross section is smaller than a minimum cross section of the second gas outlet and/or than a maximum cross section of the condensation container. The cross section of the gas line connecting piece between the second gas outlet and the condensation container can be constant. However, it is especially advantageous if the gas line connecting piece has a nozzle with a cross section that narrows in the direction from the second gas outlet toward the condensation container. The nozzle allows the operating gas to be compressed before it expands in the condensation container, improving the separating power.

It is especially advantageous if one or more areas of an inner surface of the transitional area, especially in the area of the cross-sectional narrowing, are angular and/or have swirl elements. The edges and swirl elements allow an improved condensation of at least part of the water vapor held in the operating gas.

In another advantageous embodiment of the invention, the gas line section has a first gas line section and a second gas line section, which are connected together through connecting arms of a T joint that run in a line, and the second gas outlet is formed by the part of the T joint that branches off largely perpendicular to the connecting arms. The use of a T joint simplifies the connection of the gas line section with the condensation container, especially if the condensation container is another gas line section. It is also possible to use T joints which taper or are nozzle-shaped in the transitional area at the place of the second gas outlet, and thus have an advantageous nozzle function.

In another possible embodiment of the invention, the condensation container can be a further gas line section with a gas inlet and a gas outlet. This is especially advantageous if the gas line section is connected with still other components, such as, for example, a pressure sensor, whose functionality is, under some circumstances, impaired by condensed water collecting in the components, that is, e.g., in the pressure sensor. If the further gas line section is in the form of a condensation container, the condensed water collects in the gas line section and can be discharged directly out of it, without getting into the other components, that is, e.g., the pressure sensor.

The condensation container preferably has an area with a cross section that is 10 to 100,000 times, especially 16 times, 400 times, 1,600 times, or 40,000 times greater than a cross section in the transitional area from the second gas outlet to the condensation container. Furthermore, it is advantageous if a distance between these areas is at least 50 mm, especially 50 to 100 mm or 75 mm.

A minimum diameter in the transitional area from the second gas outlet to the condensation container is preferably 0.5 to 5 mm, especially 1 mm or 2.5 mm, and a maximum diameter of the condensation container is preferably 20 to 100 mm, especially 50 mm or 75 mm.

The gas line section and/or the further gas line section can have a diameter from 5 to 10 mm, especially 6 mm or 8 mm. The gas line section and/or the further gas line section can be in the form of a tube or a plastic pipe.

The inventive device can be especially suitable for separating condensed water from an operating gas of a pneumatic pulsatile system.

Furthermore, the invention includes a pneumatic pulsatile system for a diaphragm pump of a cardiac support system with a pneumatic drive, an operating gas, and a gas line to carry the operating gas, the gas line being connectable to the diaphragm pump. The inventive pulsatile system is characterized in that the gas line is connected with an above-described device for separating condensed water from the operating gas.

The invention also includes a cardiac support system with a diaphragm pump and, connected with the diaphragm pump, a pneumatic pulsatile system as is described above.

Some examples of an inventive device for separating condensed water are described below using Figures. These examples mention various elements that are essential to the invention or also that represent advantageous further developments, some of these elements also being usable as such for further development of the invention—even outside the context of the respective example and other features of the respective example. Furthermore, in the figures, elements which are the same or similar are labeled using the same or similar reference numbers, and therefore their explanation is partly omitted.

The figures use the following reference numbers:

1 Gas line section

1a, 1b First and second gas line sections

2 Gas inlet

3, 4 First and second gas outlets

4a Cross section of second gas outlet

5 Condensation container

5a Cross section of condensation container

5b Wall

5d End

6 Gas line connecting piece

6a Cross section of gas line connecting piece

6b Nozzle

7 Condensed water

8 Water vapor

9 T joint

9a, 9b Connecting arms

9c Perpendicular part of T joint

10 Plug

11 Pneumatic drive

12 Gas line for diaphragm pump

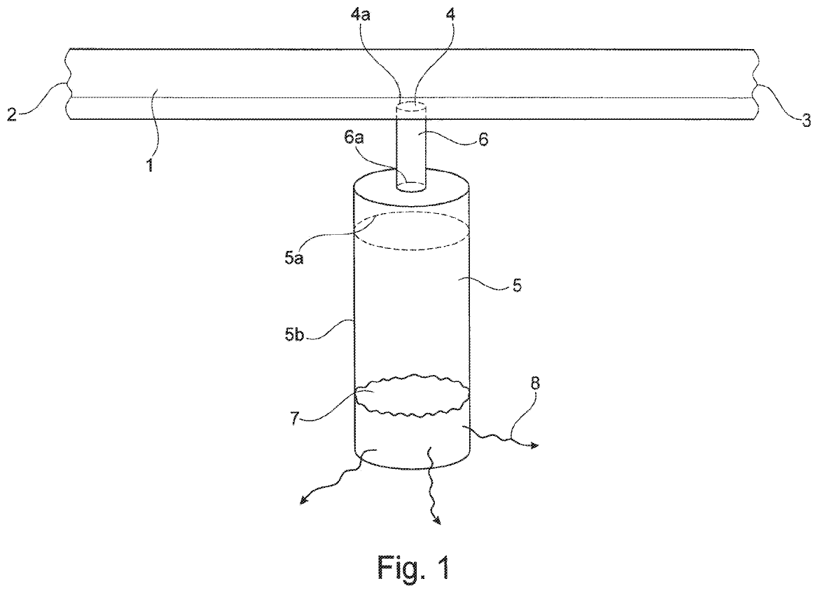

FIG. 1 shows a first sample embodiment of an inventive device for separating condensed water from the operating gas of a pneumatic pulsatile system. The device has a gas line section 1 with a gas inlet 2 and a first gas outlet 3. Gas line section 1 is part of a gas line of the pneumatic pulsatile system. On the gas inlet side, the device is connectable with a pneumatic drive (not shown). Such a pneumatic drive has, for example, a cylinder, in which a piston is moved back and forth by means of a motor, periodically producing an excess pressure and negative pressure in the gas line. In the case of a cardiac support system with a diaphragm pump, the device is connectable at the first gas outlet 3 with the diaphragm pump. Thus, the gas line section 1 communicates the excess pressure and negative pressure produced in the pneumatic drive to the membrane of the diaphragm pump. Approximately perpendicular to the longitudinal extension of gas line section 1, the gas line section 1 has a second gas outlet 4 with a cross section 4a. The second gas outlet 4 is connected, by means of a gas line connecting piece 6 having cross section 6a, with a condensation container 5 having a constant cross section 5a. The cross section 6a of the gas line connecting piece 6 corresponds to the cross section 4a of the second gas outlet 4a. The cross section 5a of the condensation container 5 is clearly greater than the cross section 6a of the gas line connecting piece 6. The condensation container 5 further has a wall 5b, that contains a semipermeable material. This material is permeable to water and/or water vapor, and is impermeable to the operating gas.

The mechanism of the first sample embodiment of the inventive device can be qualitatively described approximately as follows: If the operating gas flows through the gas line section 1, part of the operating gas goes through the second gas outlet 4 and the gas line connecting piece 6, into the condensation container 5. At the transition from the gas line connecting piece 6 to the condensation container 5, the operating gas encounters a cross-sectional enlargement, namely from the smaller cross section 6a to the clearly larger cross section 5a. This leads to a rapid expansion of the operating gas in the condensation container, and to a clear cooling of the operating gas. This causes the water vapor contained in the operating gas to condense to water 7 in the condensation container. The condensed water 7 wets the lower part of the wall 5b of the condensation container 5 and is discharged, in the form of water vapor or water, through the water-permeable wall 5b of the condensation container and out of the condensation container 5, and thus out of the pneumatic pulsatile system.

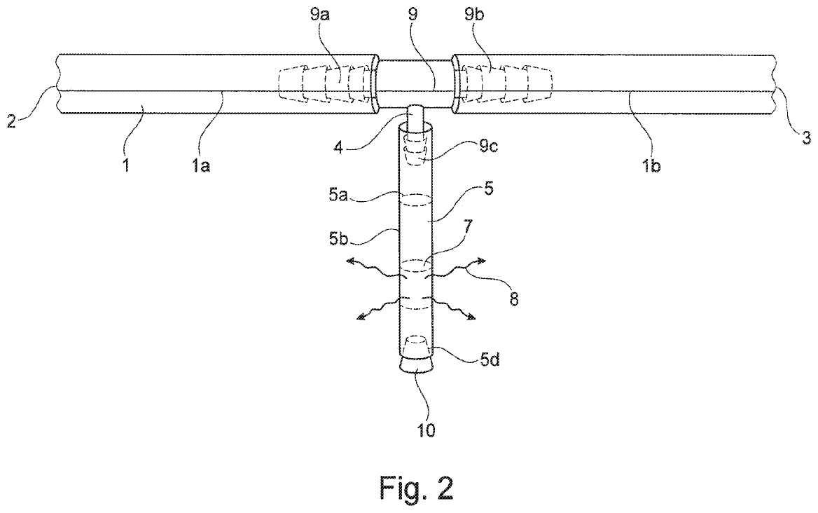

FIG. 2 shows a second sample embodiment of an inventive device for separating condensed water from a pneumatic pulsatile system. In this sample embodiment, gas line section 1 is subdivided into a first gas line section 1a and a second gas line section 1b. Gas line section 1 has a gas inlet 2 and a first gas outlet 3. The first gas line section 1a and second gas line section 1b are connected together through a T joint 9. The T joint 9 has two connection arms 9a and 9b running in a line, connection arm 9a being inserted into the first gas line section la on the gas outlet side and connection arm 9b being inserted into the second gas line section 9b on the gas inlet side. Through the T joint 9, the operating gas can flow from gas inlet 2 to the first gas outlet 3, or in the opposite direction. Furthermore, T joint 9 has a perpendicular part 9c, which is arranged essentially halfway between connection arms 9a and 9b and forms a second gas outlet 4. The perpendicular part 9c is oriented so that it allows the operating gas to flow through the second gas outlet 4, essentially perpendicular to the longitudinal extension of gas line section 1. The perpendicular part 9c is further connected with another gas line section 5, which forms a condensation container. The further gas line section 5 is closed with a plug 10 at its end 5d facing away from the gas line section 1. Alternatively, it is conceivable to connect a pressure sensor at the end 5d instead of the plug 10. Starting from gas outlet 4, the perpendicular part 9c has a tapering cross section, and therefore acts as a nozzle which can intensify the condensation effect in the further gas line section 5. As in the first sample embodiment, the cross section 5a of the condensation container is clearly greater than the cross section at the second gas outlet 4 and in the rest of the perpendicular part 9c. The further gas line section has a wall 5b, that contains a semipermeable material. This material is permeable to water and/or water vapor, and impermeable to the operating gas.

The mechanism of the second sample embodiment of the device can be qualitatively described approximately as follows: Part of the operating gas flowing through gas line section 1 goes through the second gas outlet 4 into the perpendicular part 9c of the T joint 9. The tapering cross section of the perpendicular part 9c compresses this part of the operating gas in the perpendicular part 9c. This compression allows part of the water vapor held in the operating gas to condense in the form of water and go into the further gas line section 5, which serves as a condensation container. At the end of the perpendicular part 9c of the T joint 9, when the operating gas goes into the further gas line section 5, it encounters a cross-sectional enlargement, causing it to expand and cool rapidly. The cooling causes another part of the water vapor held in the operating gas to condense and be collected in the further gas line section 5. The collected condensed water 7 can be discharged, in the form of water vapor 8 or water, through the semipermeable wall 5b out of the further gas line section 5, and thus out of the pneumatic pulsatile system, before it can get to a pressure sensor that might be connected at the end 9d.

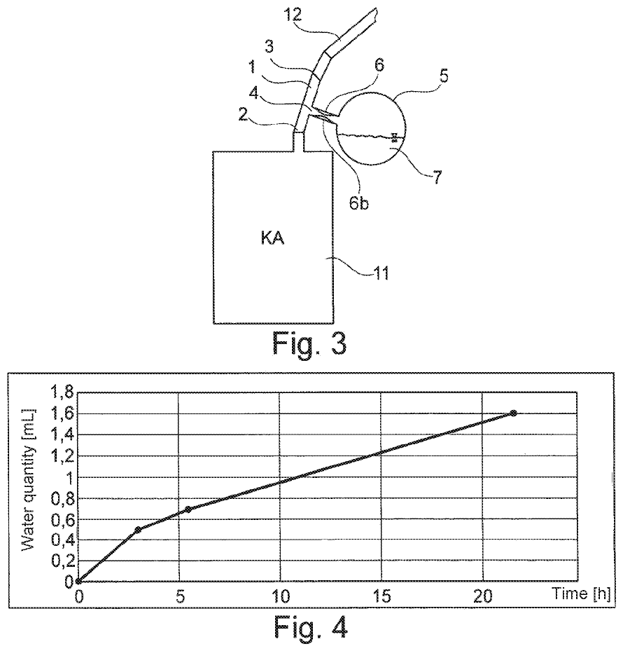

FIG. 3 schematically illustrates a test setup for proving the efficacy of the inventive device in a cardiac support system with a diaphragm pump. The test setup shown comprises only the drive line of the cardiac support system. The gas line section 1 is connected, at its gas inlet 2, with a pneumatic drive 11. At its first gas outlet 3, it is attached to a gas line 12, with which the diaphragm pump is connected. The first gas line section 1 together with the gas line 12 are also referred to as a drive tube. At the second gas outlet 4, a condensation container 5 is attached through a gas line connecting piece 6. Here the condensation container 5 is shown in the form of a spherical container. The gas line connecting piece 6 has a nozzle 6b. Furthermore, air serves as the operating gas in the test setup. Every stroke of the piston working in the pneumatic drive 11 presses some humid air through the nozzle 6b, and the air then expands. In the process, the water stored in the air condenses and collects in the condensation container 5. This allows dehumidification of the air in the drive tube 1, 12.

For the test, the pneumatic drive 11 is operated in a circulation model with a diaphragm pump having a displacement of 80 mL. After a warm-up period of about 2 h, distilled water is put into the drive tube 1, 12 through a syringe. In the test setup used, the nozzle 6b of the gas line connecting piece is formed by a Luer adapter. The condensation container 5 is formed by a syringe connected with the Luer adapter, the position of the piston of the syringe having been fixed.

To determine the amount of condensed water that has been separated, the condensation container is removed from the Luer adapter and weighed. The weight before the beginning of the test was 40.0 g. The time behavior of the separated amount of water is shown in FIG. 4.

After a time of 21.5 h, the test was ended. The amount of water separated was 1.6 mL, and the drive tube 1, 12 was completely dry. Thus, the separation rate is >1.8 mL per day.

This confirms the efficacy of the condensed water separation through a nozzle and an attached condensation container.

The invention further comprises the following aspects:
1. A device for separating condensed water from an operating gas of a pneumatic pulsatile system, the device having
   a gas line section with a gas inlet and a first and a second gas outlet,
   characterized in that
   the device comprises a condensation container for extracting the condensed water from the operating gas, this condensation container being connected with the second gas outlet, a transitional area from the second gas outlet to the condensation container having a cross-sectional enlargement, and
   the condensation container has means for discharging the extracted condensed water.
2. A device according to the preceding aspect, characterized in that the means for discharging the extracted condensed water that the condensation container has are in the form of a water-permeable material, especially a semipermeable plastic, a fluoropolymer and/or a sintered metal.
3. A device according to any one of the preceding aspects, the transitional area from the second gas outlet to the condensation container having a cross-sectional narrowing before the cross-sectional enlargement.
4. A device according to any one of the preceding aspects, wherein a gas line connecting piece is arranged between the second gas outlet and the condensation container, a minimum cross section of the gas line connecting piece being smaller than a minimum cross section of the second gas outlet and/or than a maximum cross section of the condensation container.
5. A device according to the preceding aspect, characterized in that the cross section of the gas line connecting piece between the second gas outlet and the condensation container is constant.
6. A device according to aspect 4, characterized in that the gas line connecting piece has a nozzle with a cross section that narrows in the direction from the second gas outlet toward the condensation container.
7. A device according to any one of the preceding aspects, characterized in that the gas line section has a first gas line section and a second gas line section, which are connected together through connecting arms of a T joint that run in a line, and the second gas outlet is formed by the part of the T joint that branches off largely perpendicular to the connecting arms.
8. A device according to any one of the preceding aspects, wherein the condensation container is a further gas line section with a gas inlet and a gas outlet.
9. A device according to the preceding aspect, characterized in that the gas outlet of the further gas line section is connected with a pressure sensor.
10. A device according to any one of the preceding aspects, characterized in that the condensation container has an area with a cross section that is 10 to 100,000 times, especially 16 times, 400 times, 1,600 times, or 40,000 times greater than a cross section in the transitional area from the second gas outlet to the condensation container, and in that a distance between these areas is at least 50 mm, especially 50 to 100 mm or 75 mm.
11. A device according to any one of the preceding aspects, characterized in that a minimum diameter in the transitional area from the second gas outlet to the condensation container is 0.5 to 5 mm, especially 1 mm or 2.5 mm, and a maximum diameter of the condensation container is 20 to 100 mm, especially 50 mm or 75 mm.
12. A device according to any one of the preceding aspects, characterized in that the gas line section and/or the further gas line section has a diameter from 5 to 10 mm, especially 6 mm or 8 mm.
13. A device according to any one of the preceding aspects, characterized in that the gas line section and/or the further gas line section is a tube or a plastic pipe.
14. A pneumatic pulsatile system for a diaphragm pump of a cardiac support system with a pneumatic drive, an operating gas, and a gas line to carry the operating gas, the gas line being connectable to the diaphragm pump, characterized in that the gas line is connected with a device according to any one of the preceding aspects.
15. A cardiac support system with a diaphragm pump and a pneumatic pulsatile system according to the preceding aspect that is connected with the diaphragm pump.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, or N including any one element alone or the one element in combination with one or more of the other elements which may also include, in combination, additional elements not listed. Unless otherwise indicated or the context suggests otherwise, as used herein, “a” or “an” means “at least one” or “one or more.”

1. A device for separating condensed water from an operating gas of a pneumatic system, comprising:
a gas line section that is connected with the pneumatic system and that has a gas inlet and a first and a second gas outlet; and
a condensation container for extracting the condensed water from the operating gas and for collecting the condensed water, this condensation container being connected with the second gas outlet;
wherein
to discharge the condensed water, the condensation container has a water-permeable material.

2. The device of claim 1, wherein a transitional area from the second gas outlet to the condensation container has a cross-sectional enlargement.

3. The device of claim 2, wherein the transitional area from the second gas outlet to the condensation container has a cross-sectional narrowing before the cross-sectional enlargement.

4. The device of claim 1, wherein a gas line connecting piece is arranged between the second gas outlet and the condensation container, a minimum cross section of the gas line connecting piece being smaller than a minimum cross section of the second gas outlet and/or than a maximum cross section of the condensation container.

5. The device of claim 4, wherein the cross section of the gas line connecting piece between the second gas outlet and the condensation container is constant.

6. The device of claim 4, wherein the gas line connecting piece has a nozzle with a cross section that narrows in the direction from the second gas outlet toward the condensation container.

7. The device of claim 1, wherein the gas line section has a first gas line section and a second gas line section, which are connected together through connecting arms of a T joint that run in a line, and the second gas outlet is formed by the part of the T joint that branches off largely perpendicular to the connecting arms.

8. The device of claim 1, wherein the condensation container is a further gas line section with a gas inlet and a gas outlet.

9. The device of claim 8, wherein the gas outlet of the further gas line section is connected with a pressure sensor.

10. The device of claim 2, wherein the condensation container has an area with a cross section that is 10 to 100,000 times greater than a cross section in the transitional area from the second gas outlet to the condensation container, and in that a distance between these areas is at least 50 mm.

11. The device of claim 2, wherein a minimum diameter in the transitional area from the second gas outlet to the condensation container is 0.5 to 5 mm, and a maximum diameter of the condensation container is 20 to 100 mm.

12. The device of claim 1, wherein the gas line section and/or the further gas line section has a diameter from 5 to 10 mm.

13. The device of claim 1, wherein the gas line section and/or the further gas line section is a tube or a plastic pipe.

14. The device of claim 1, wherein the device configured to separate condensed water from an operating gas of a pneumatic pulsatile system.

15. A pneumatic pulsatile system for a diaphragm pump of a cardiac support system comprising a pneumatic drive, an operating gas, and a gas line to carry the operating gas, the gas line being connectable to the diaphragm pump, wherein the gas line is connected with a device comprising:
a gas line section that is connected with the pneumatic system and that has a gas inlet, a first gas outlet, and a second gas outlet, wherein the first gas outlet is connected to the gas line and the gas inlet is connected to the pneumatic drive; and
a condensation container for extracting the condensed water from the operating pas and for collecting the condensed water, this condensation container being connected with the second gas outlet, wherein to discharge the condensed water, the condensation container has a water-permeable material.

16. The pneumatic pulsatile system of claim 15 further comprising the diaphragm pump, which is connected to the gas line.

17. The device of claim 1, wherein the water-permeable material includes a semipermeable plastic, a fluoropolymer, and/or a sintered metal.
